(19) United States
(12) Patent Application Publication
(10) Pub. No.: US 2026/0324613 A1
(43) Pub. Date: Oct. 22, 2026

(54) NEW COMPOSITION FOR USE IN THE TREATMENT OF DISEASES OF THE FEET IN HOOFED OR HOOF-BEARING ANIMALS AND A DEVICE THEREFORE

(71) Applicant: BD EQUIPMENT APS, Haderslev (DK)

(72) Inventor: Bjørn Drews ØSTEDGAARD, Haderslev (DK)

(73) Assignee: BD EQUIPMENT APS, Haderslev (DK)

(21) Appl. No.: 19/491,434

(22) PCT Filed: Jun. 10, 2024
(86) PCT No.: PCT/IB2024/000270

§ 371 Date: Dec. 9, 2025

(30) Foreign Application Priority Data
Jun. 9, 2023 (DK) PA202330075

(51) Int. Cl. A01L 15/00; A61K 9/00; A61K 31/60; A61K 47/34; A61L 24/00

(52) U.S. Cl. CPC A01L 15/00; A61K 9/0014; A61K 31/60; A61K 47/34; A61L 24/0015

(57) ABSTRACT

Herein is detailed the use of a water-dispersed polyvinyl acetate-based adhesive as an active substance in the treatment of digital dermatitis in hoofed and hoof-bearing animals, as well as a carrier for a medically active substance, in which the medically active substance is salicylic acid, a composition comprising a water-dispersed polyvinyl acetate-based adhesive and salicylic acid, and the use of the composition for the treatment of digital dermatitis in hoofed or hoof-bearing animals, preferably for cattle.

### FIELD OF THE INVENTION

Within the treatment of foot diseases in hoofed or hoof-bearing animals, new compositions are described which are effective in improving the healing of foot diseases in hoofed or hoof-bearing animals, particularly in cattle, in which the composition consists of a water-dispersed polyvinyl acetate-based adhesive, preferably supplemented with a substance, that is medically active against the treated foot disease. In a preferred embodiment, the medicinally active substance is salicylic acid, and the foot disease is digital dermatitis in cattle.

### BACKGROUND

The presence of, and the necessary treatment of, foot diseases in domesticated hoofed or hoof-bearing animals, such as cattle and pigs, is a significant economic, as well as animal welfare, problem for the livestock industry. The resulting problems include not only the direct cost of treating the foot diseases, but also the deterioration of livestock performance and consequent loss of income for the farmer. Added to this are the animal ethical issues, as the prerequisite for keeping livestock in Denmark for meat and milk production is consumers' acceptance of, and trust in, that the farmer adheres to good animal ethics and does not inflict unnecessary suffering on the animals.

The combination of society's economic need for industrialized livestock farming, and the resulting issues around stabling and production optimization, has led, for example, to dairy cows, especially dairy cows belonging to high-performing dairy breeds, not fully exploiting their production potential due to various cattle diseases, such as e.g. lameness.

Lameness, which is a general, non-specific diagnostic term within the industry, occurs in the individual COW as a consequence of one or more diseases, either infectious or non-infectious, such as, for example, sole ulcers/crushing (Pododermatitis circumscripta), digital dermatitis, bale rot (Erosio ungulae), neoplasms (Limax), foot abscess (Phlegmona interdigitalis), foot abscess (Pododermatitis) and/or cleft inflammation.

In a previous patent application (WO 2023/031671), the present inventor has described devices for local irrigation with medicinal liquids, with which, for example, the above-mentioned diseases in cattle and other hoofed or hoof-bearing animals can be advantageously treated medically with local irrigation with a healing medicinal liquid designed for this purpose, rather than with previously used global rinses (hoof baths) or local bandages, and the associated practical and medical limitations.

The present patent application thus describes new compositions that are effective for the use of medical treatment by local irrigation, for example by local irrigation using one of the devices described in WO 2023/031671.

It is also an advantage of the present invention that, with small modifications, the portable device herein detailed can also be used by other hoofed or hoof-bearing livestock in need of the treatments mentioned herein, such as cattle, pigs, sheep, goats, horses, donkeys, mules, camels, dromedaries, and/or reindeer. All these domestic animals can experience the diseases described herein to varying degrees and can therefore benefit from the compositions described herein when these are suitably adapted to the physiology of the individual domestic animal.

### SHORT DESCRIPTION OF THE FIGURES

FIG. 1: Before (A) and after (B) photography of hindleg with digital treated according to the invention using PVAc. After removal (by scraping) of treated digital (C).

FIG. 2: Before (A) and after (B) photography of hindleg with digital treated according to the invention using PVAc plus salicylic acid. After removal (by scraping) of treated digital (C).

FIG. 3: PVAc-treated digital (A) after scraping of treated digital. PVAc plus salicylic acid treated digital (B) after scraping of treated digital.

FIG. 4: Pouch for hoof application for use in the treatment of digital dermatitis, seen hoof-facing (4A), hoof off-facing (4B) and perpendicular to a domestic animal's hoof (4C).

### DETAILED DESCRIPTION

This invention relates in a first aspect and embodiment to the use of salicylic acid in liquid form for the treatment of digital dermatitis in hoofed or hoof-bearing livestock.

Likewise, this invention relates to salicylic acid as liquid formulation for the treatment of digital dermatitis in hoofed or hoof-bearing livestock.

In the prior art, salicylic acid is used in the treatment of digital dermatitis only in powder form, but when using the device described herein for application to a hoofed or hoof-bearing domestic animal, this limitation is no longer necessary. On the contrary, the experiments show that liquid formulated salicylic acid has medical efficiency without the skin damage normally characteristic of salicylic acid, such as fragile and porous newly formed skin in the healing area.

In a preferred embodiment of this, salicylic acid is mixed with a water-dispersed polyvinyl acetate-based adhesive.

In a further preferred embodiment of this, the hoofed or hoof-bearing domestic animal is cattle.

Likewise, this invention relates in another aspect to the use of a water-dispersed polyvinyl acetate-based adhesive as a carrier for a medically active substance.

In a preferred embodiment of this, the medically active substance is salicylic acid.

In a preferred embodiment of this, a use of a water-dispersed polyvinyl acetate-based adhesive as a carrier for salicylic acid is described, for the treatment of digital dermatitis in a hoofed or hoof-bearing domestic animal.

Likewise, in a preferred embodiment of this, a water-dispersed polyvinyl acetate-based adhesive is described as a carrier for salicylic acid, for the treatment of digital dermatitis in a hoofed or hoof-bearing domestic animal.

In a preferred embodiment of this, the hoofed or hoof-bearing domestic animal is cattle.

Furthermore, this invention relates in one aspect to the use of a water-dispersed polyvinyl acetate-based adhesive for the treatment of digital dermatitis in hoofed or hoof-bearing livestock.

Likewise, this invention relates in one aspect to a water-dispersed polyvinyl acetate-based adhesive for the treatment of digital dermatitis in hoofed or hoof-bearing livestock.

In a preferred embodiment of this, it is described that salicylic acid is mixed with said water-dispersed polyvinyl acetate-based adhesive.

In a preferred embodiment of this, it is described that the hoofed or hoof-bearing domestic animal is cattle.

In embodiments of all the above-mentioned aspects and embodiments thereof, the water-dispersed polyvinyl acetate-based adhesive is a polyvinyl acetate-based adhesive which, after curing, can be characterized as water-resistant in class D1 according to DIN EN 204, 2016-11. By having a low hardening class according to DIN EN 204, such as D1, it is avoided that any adhesive residues left during the treatment harden without being able to be washed away with water.

In embodiments of all the above-mentioned aspects and embodiments thereof, the pH of the water-dispersed polyvinyl acetate-based adhesive is from pH 2.5 to pH 6, preferably from pH 3.5 to pH 5.

In embodiments of all the above-mentioned aspects and embodiments thereof, the solids content of polyvinyl acetate in the water-dispersed polyvinyl acetate-based adhesive is from 35 w/w % to 55 w/w %, preferably from 40 w/w % to 50 w/w% , more preferably from 42.5 w/w % to 47.5 w/w %.

In embodiments of all the above-mentioned aspects and embodiments thereof, said water-dispersed polyvinyl acetate-based adhesives are mixed with salicylic acid in a concentration of up to 15 w/w %, preferably up to 10 w/w %, more preferably up to 5 w/w %, and most preferably up to 2.5 w/w %.

Likewise, this invention relates in a further aspect to a composition comprising a water-dispersed polyvinyl acetate-based adhesive, preferably a water-dispersed polyvinyl acetate-based adhesive and salicylic acid.

In a preferred embodiment thereof, this invention relates to a composition comprising a water-dispersed polyvinyl acetate-based adhesive, preferably a water-dispersed polyvinyl acetate-based adhesive and salicylic acid, for the treatment of digital dermatitis in a hoofed or hoof-bearing livestock, preferably in which the livestock is cattle.

In an embodiment thereof, the present invention concerns a composition comprising a water-dispersed polyvinyl acetate-based adhesive and salicylic acid, in which salicylic acid is mixed with said water-dispersed polyvinyl acetate-based adhesive in a concentration of up to 15 w/w %, preferably up to 10 w/w %, more preferably up to 5 w/w %, and most preferably up to 2.5 w/w %, for the treatment of digital dermatitis in a hoofed or hoof-bearing livestock, preferably wherein the livestock is cattle. In one embodiment, the admixture amount is 1 w/w %.

In an embodiment thereof, the present invention relates to a composition comprising a water-dispersed polyvinyl acetate-based adhesive and salicylic acid, in which the solids content of polyvinyl acetate in the water-dispersed polyvinyl acetate-based adhesive is from 35 w/w % to 55 w/w %, preferably from 40 w/w % to 50 w/w %, more preferably from 42.5 w/w % to 47.5 w/w %.

In an embodiment thereof, the present invention relates to a composition comprising a water-dispersed polyvinyl acetate-based adhesive and salicylic acid, in which the pH of the composition is from pH 2.5 to pH 6, preferably from pH 3.5 to PH 5.

In a preferred embodiment thereof, the present invention relates to a composition comprising a water-dispersed polyvinyl acetate-based adhesive and salicylic acid for the treatment of digital dermatitis in a hoofed or hoof-bearing livestock, preferably in which the livestock is cattle.

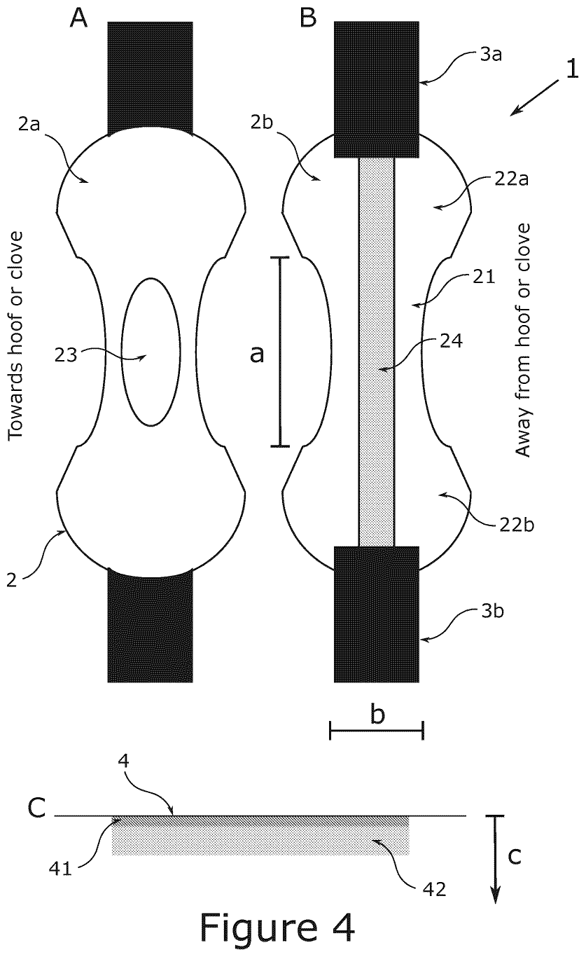

In a further aspect of the present invention (see FIG. 4), an device (1) is described for attachment around a hoof or a clove of a corresponding hoof or hoof-bearing domestic animal, which device (1) comprises a bag (2) at least enclosing a volume (abc), in which the width (b) is determined so that said bag (2) on a stretch thereof (21) determined by the length (a) can be fitted between bi-hooves, respective crown edge, and a corresponding respective hoof or clove, on said livestock; where, on said bag (2), one or more means (3a, 3b) are attached to secure said bag (2) around said hoof or clove of said livestock; therein characterized that said bag (2) defines a hoof-or clove-facing side (2a) in which an opening (23) in said bag (2) is arranged on the stretch (21), behind which opening (23) in said bag (2) to said enclosed volume (abc) is arranged a contact membrane (4) that allows a liquid contained in said enclosed volume (abc) to pass said contact membrane (4) when said device (1) is attached to and in contact with said livestock.

The portable device (1) described herein differs from the portable device known from WO 2023/031671, FIG. 4, in that said bag (2) defines a hoof-or clove-facing side (2a) (cf. FIG. 4A) in which, on a stretch (21) an opening (23) is arranged in said bag (2), behind which opening (23) to the enclosed volume (abc) of said bag (2) a contact membrane (4) (cf. FIG. 4c) is arranged, which allows a liquid contained in said enclosed volume (abc) to pass said contact membrane (4) when said device (1) is attached to and in contact with said livestock.

Thereby, a particularly compact device is achieved which can be placed and fastened to a hoof or a clove without the animal's movements leading to limitations in the discharge of liquid to a digital dermatitis on the hoof or clove. In fact, it has been shown in preliminary tests, that said contact membrane (4) can be adjusted so that when the domestic animal is at rest, said contact membrane (4) is not soaked by the liquid contained in said enclosed volume (abc), while liquid is discharged when said livestock is in motion.

Such contact membranes are well known and follow a general flow equation, where the flow across the membrane is proportional to d (P₀+ΔP)/dt, where P₀ is a threshold pressure below which flow cannot take place. P₀ is essentially determined as the osmotic balance pressure (Pₐₜₘ-Pᵢₙₜₑᵣₙₐₗ) across the contact membrane. It has been advantageously shown that movement of the livestock is sufficient to cause a pressure that is greater than P₀, whereby liquid can flow through a contact membrane mentioned herein during the movement of the livestock, while at rest the pressure is less than P₀ (due to hoof and hoof-bearing animal anatomy that leaves the hoof or clove unstressed at rest)

In an embodiment thereof, said contact membrane (4) comprises a contact membrane (41) facing said hoof or clove-facing side (2a) and a sponge (42). Thereby it is achieved that as said enclosed volume (abc) is emptied of a given liquid, a flow direction is created inside said enclosed volume (abc) such that the internal pressure (Pᵢₙₜₑᵣₙₐₗ) that said contact membrane (41) experiences essentially remains constant throughout the use of the device (1) and determined by the absorbency of said sponge (42) for said liquid.

In an embodiment of said device (1), a bias (24), preferably in the form of an elastic band, is arranged on a reverse side (2b) (cf. FIG. 4B) opposite to the side facing the hoof or clove (2a) (24), which bias is at rest (the state shown in FIG. 4B) when said device (1) is not attached around a hoof or clove of a domestic animal, and applying a tension, preferably an elongation, when said device (1) is attached on said livestock. This creates a pressure directed against the animal's hoof or clove, and the flow over the membrane increases further. Such a bias (24) is advantageous both by increasing the emptying of liquid from said device (1) during use, and by ensuring a flow of liquid towards the domestic animal, also when the domestic animal is at rest, e.g. when the domestic animal is lying down.

In a further embodiment thereof, said bag (2) comprises at least one wing (22a, 22b) arranged outside an area (ab) between bi-hoof or bi-clove and corresponding hoof or clove of said domestic animal, which wing (22a, 22b) encloses a volume in fluid contact with said enclosed volume (abc), such that the enclosed volume of said bag (2) becomes greater than said enclosed volume (abc).

In a further embodiment of said device (1), said device (1) comprises a removable closure (not shown) over said opening (23), whereby said contact membrane (24) is prevented from releasing a liquid contained in the bag (2) before desired.

Likewise, a skin-acceptable adhesive edge can be arranged around said opening (23) (not shown) to increase the contact effect between the animal's skin and said device (1). Such skin-acceptable adhesives are well-known within the field, e.g. polyacrylate adhesives, and it is considered well-known for the person skilled in the art to procure and apply such an adhesive edge when the herein instructions are followed.

In a further aspect of the present invention, a kit-of-parts is herein detailed comprising an device (1) for attachment around a hoof or a clove of a corresponding hoof or hoof-bearing domestic animal according to any of the embodiments described herein, wherein said enclosed volume (abc) comprises a composition comprising a water-dispersed polyvinyl acetate-based adhesive, preferably a water-dispersed polyvinyl acetate-based adhesive and salicylic acid, for the treatment of digital dermatitis in a hoof or hoof-bearing domestic animal according to any of the above-mentioned embodiments, preferably wherein the livestock is cattle.

### EKSPERIMENTAL

### Experimental Setup

In an initial trial, 8 cattle from a cross-bred herd with genetic origin Red Holstein, Black-spotted Danish Dairy Cow, and Jersey were selected after milking the day before the test trial was run. The criterion for selection was the presence of digitals (D.D.) on both hind legs.

For the test, a portable device was used in accordance with WO 2023/031671 (cf. FIG. 4 and associated description), and filled with a volume which, for the relevant nozzle size of the outlet nozzle, was adapted to deliver a liquid stream of the water-dispersed polyvinyl acetate in this in vivo experiment under the influence of gravity for 4 days, corresponding to the standard treatment length for salicylic acid bandage treatment.

Two of the cattle were treated with a traditional bandage treatment with salicylic acid approx. 30 g of salicylic acid per digital.

Three of the animals were treated with the water-dispersed polyvinyl acetate-based adhesive alone, while the last three animals were treated with 10 g of salicylic acid dissolved in the aqueous phase of the water-dispersed polyvinyl acetate-based adhesive. Elmers Skolelim 946 ml, total weight 1012 g, concentration of salicylic acid 10 g/1022 g i.e. approx. 1 w/w %.

The water-dispersed polyvinyl acetate-based adhesive used for the experiments was of the brand Elmers Skolelim; pH 3.5-5, specific gravity 1.07 g/ml; solids content in wt %, 47 w/w %, at STP conditions.

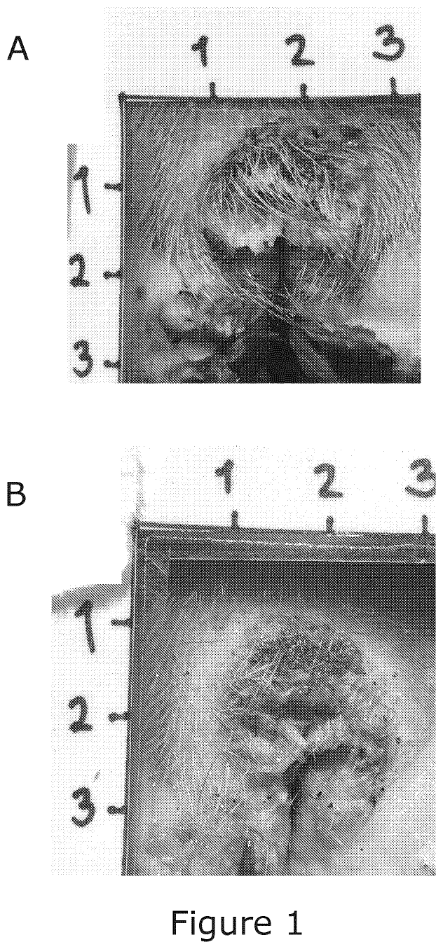

Before the experiments were started, the animals were pacified in a hoof box and had the digitals washed with lukewarm water, and the digitals were photographed and measured, see FIG. 1. The treatment lasted over 4 days (standard length of treatment for salicylic acid bandage treatment), after which the animals were again pacified in a hoof box, the digitals were washed with lukewarm water, after which the digitals were re-photographed and measured.

### Results

Test group 1—Elmers Skolelim

In the treated area, the digitals die, and the skin heals (see FIG. 1). Smaller digitals died completely, while larger digitals became smaller, which corresponds to the expectation of similar treatment with bandage.

As shown in FIG. 1, an aqueous solution of PVAC, pH 3.5-5, is in itself effective in the treatment of digital dermatitis in cattle.

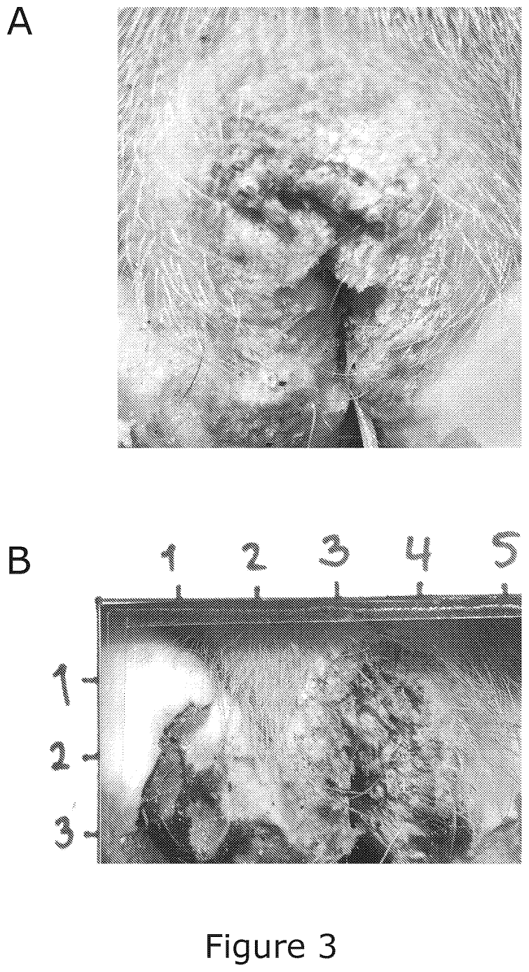

When scraping away treated (dead) digitals, it is seen (FIG. 3A) that the skin is normally firm and healthy (pink) in color.

### Test Group 2—Elmers Skolelim With Salicylic Acid

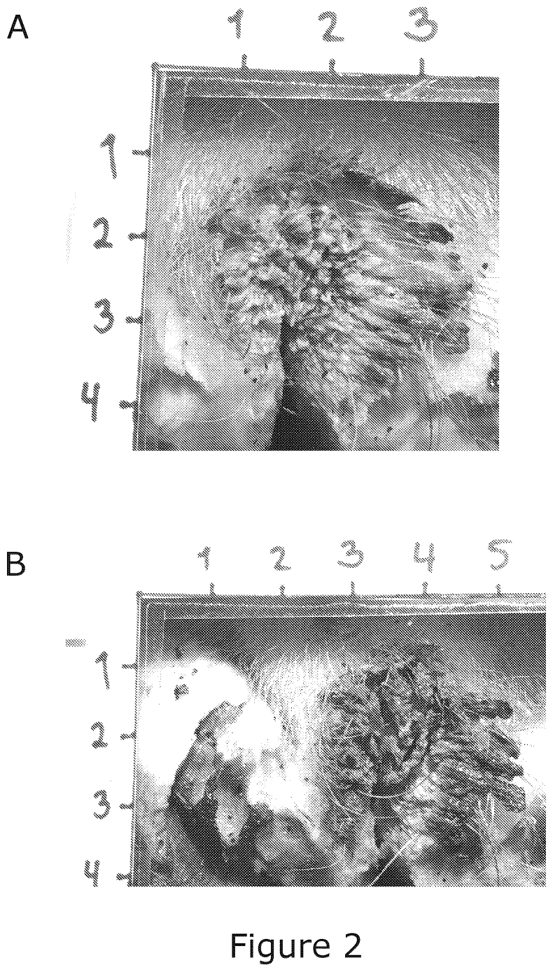

In the treated area, the digitals die, and the skin heals (see FIG. 2). Smaller digitals died completely, while larger digitals became smaller, which corresponds to the expectation of similar treatment with bandage.

When scraping away treated (dead) digitals it is seen (FIG. 3B) that the skin is normally firm and healthy (pink) in color.

In contrast to the comparison experiment with salicylic bandages (Test group 3), the treated skin does not become neither porous nor fragile after four days of treatment with the salicylic acid-containing aqueous solution of PVAC.

The test indicated that the presence of salicylic acid at low concentration enhances the clinical effect of the treatment with the water-dispersed polyvinyl acetate-based adhesive, even at a concentration as low as 1 w/w % dissolved salicylic acid.

Further, preliminary tests (not shown) indicate that addition of salicylic acid up to 15 w/w % has an enhancing effect on the treatment of digital dermatitis but without harming the treated skin of the cattle.

### Test Group 3—Salicylic Acid Bandage

As expected, the digitals die, smaller digitals completely, while larger digitals are reduced in size.

When scraped, the skin is porous and fragile after four days of exposure to close contact with salicylic acid, corresponding to well-known side effects of salicylic acid bandage treatment.

### Conclusion

The test result showed that the water-dispersed polyvinyl acetate adhesive in itself was active against digital dermatitis infection in the treated cattle, and thereby demonstrated a clinical effect.

The experiments further showed that salicylic acid in aqueous form, applied to the treatment area using the device according to WO 2023/031671, FIG. 4, in a one-third dilution compared to the control treatment with classic salicylic acid bandages, was equally effective in the treatment of digital dermatitis in the studied animals compared to classic salicylic acid bandages, but significantly gentler compared to the salicylic acid bandages.

Likewise, the tests showed that the water-dispersed polyvinyl acetate-based adhesive was suitable for ensuring that the salicylic acid came into contact with the area requiring treatment and at the same time ensuring that the liquid did not flow away from the treatment requiring area faster than new liquid could flow in from the device according to WO 2023/031671, FIG. 4.

1-19. (canceled)

20. A device for attachment around a hoof or a clove of a corresponding hoof or hoof-bearing domestic animal, which device comprises a bag at least enclosing a volume, in which the width is determined so that said bag on a stretch thereof determined by the length can be fitted between bi-hooves, respective crown edge, and a corresponding respective hoof or clove, on said livestock; where, on said bag, one or more means are attached to secure said bag around said hoof or clove of said livestock; wherein said bag defines a hoof- or clove-facing side in which an opening in said bag is arranged on the stretch, behind which opening in said bag to said enclosed volume is arranged a contact membrane that allows a liquid contained in said enclosed volume to pass said contact membrane when said device is attached to and in contact with said livestock.

21. The device for attachment around a hoof or a clove on a corresponding hoof or hoof-bearing domestic animal according to claim 20, wherein said contact membrane comprises a contact membrane facing said hoof- or clove-facing side and a sponge.

22. The device for attachment around a hoof or a hoof of a corresponding hoof or hoof-bearing domestic animal according to claim 20, wherein said bag comprises at least one wing arranged outside an area between bi-hoof or bi-clove and corresponding hoof or clove of said livestock, which wing encloses a volume in fluid contact with said enclosed volume such that said enclosed volume becomes greater than said enclosed volume.

23. The device for attachment around a hoof or a clove of a corresponding hoof or hoof-bearing domestic animal according to claim 20, wherein said bag comprises, on a back side opposite the hoof or clove-facing side, an arranged bias, which bias is at rest when said device is not attached around a hoof or a clove of a domestic animal, and applied with a tension, when said device is attached to said domestic animal.

24. The device for attachment around a hoof or clove of a corresponding hoof or hoof-bearing domestic animal according to claim 20, further comprising a removable closure over said opening.

25. The device for attachment around a hoof or clove of a corresponding hoof or hoof-bearing domestic animal according to claim 20, further comprising a skin-acceptable adhesive edge arranged around said opening.

26. A kit-of-parts comprising a device for attachment around a hoof or a clove of a corresponding hoof or hoof-bearing domestic animal according to claim 20, wherein said enclosed volume comprises a composition comprising a water-dispersed polyvinyl acetate-based adhesive for the treatment of digital dermatitis in a hoof or hoof-bearing domestic animal.

27. The kit-of-parts according to claim 26, wherein said composition comprises a water-dispersed polyvinyl acetate-based adhesive and salicylic acid.

28. The kit-of-parts according to claim 26, wherein said domestic animal is cattle.

29. The kit-of-parts according to claim 26, wherein the pH of the water-dispersed polyvinyl acetate-based adhesive is from pH 2.5 to pH 6.

30. The kit-of-parts according to claim 26, wherein the solids content of polyvinyl acetate in the water-dispersed polyvinyl acetate-based adhesive is from 35 w/w % to 55 w/w %.

31. The kit-of-parts according to claim 26, wherein the water-dispersed polyvinyl acetate-based adhesive is a polyvinyl acetate-based adhesive which, after curing, can be characterized as water resistant in class D1 according to DIN EN 204, 2016-11.

32. The kit-of-parts according to claim 27, wherein salicylic acid is mixed with said water-dispersed polyvinyl acetate-based adhesives in a concentration of up to 15 w/w %.

33. A composition comprising a water-dispersed polyvinyl acetate-based adhesive and salicylic acid.

34. The composition comprising a water-dispersed polyvinyl acetate-based adhesive and salicylic acid according to claim 33, wherein the pH of the composition is from pH 2.5 to pH 6.

35. The composition comprising a water-dispersed polyvinyl acetate-based adhesive and salicylic acid according to claim 33, wherein the solids content of polyvinyl acetate in the water-dispersed polyvinyl acetate-based adhesive is from 35 w/w % to 55 w/w %.

36. The composition comprising a water-dispersed polyvinyl acetate-based adhesive and salicylic acid according to claim 33, wherein the water-dispersed polyvinyl acetate-based adhesive is a polyvinyl acetate-based adhesive which, after curing, can be characterized as water resistant in class D1 according to DIN EN 204, 2016-11.

37. The composition comprising a water-dispersed polyvinyl acetate-based adhesive and salicylic acid according to claim 33, wherein salicylic acid is mixed with said water-dispersed polyvinyl acetate-based adhesives in a concentration of up to 15 w/w %.
